(19) United States
(12) Patent Application Publication
(10) Pub. No.: US 2006/0079545 A1
(43) Pub. Date: Apr. 13, 2006

(54) Palonosetron for the treatment of chemotherapy induced emeses

(75) Inventors: Alberto Macciocchi, Taverne (CH); Sergio Cantoreggi, Vezia (CH); Enrico Braglia, Pazzallo (CH); Riccardo Braglia, Pazzallo (CH); Simone Macciocchi, (US); Giulio Macciocchi, (US)

Correspondence Address: KING & SPALDING LLP, 191 PEACHTREE STREET, N.E., 45TH FLOOR, ATLANTA, GA, 30303-1763, US

(73) Assignee: Helsinn Healthcare SA, Lugano (CH)

(21) Appl. No.: 11/129,839

(22) Filed: May 16, 2005

Related U.S. Application Data
(63) Continuation-in-part of application No. PCT/IB03/05567, filed on Nov. 6, 2003.
(60) Provisional application No. 60/426,829, filed on Nov. 15, 2002.

(51) Int. Cl. A61K 31/4745

(52) U.S. Cl. 514/296

(57) ABSTRACT

Methods and compositions for reducing chemotherapy and radiotherapy induced emises with 5-HT₃ receptor antagonists are disclosed, especially with palonosetron.

### RELATED APPLICATIONS

This application is a continuation-in-part of PCT application no. IB03/005567, filed Nov. 6, 2003, which claims priority to U.S. provisional application No. 60/426,829, filed Nov. 15, 2002.

### FIELD OF THE INVENTION

The present invention relates to methods for reducing chemotherapy and radiotherapy induced emesis with 5-HT₃ receptor antagonists. In particular, the invention relates to methods for reducing chemotherapy and radiotherapy induced emesis with palonosetron.

### BACKGROUND OF THE INVENTION

Emesis is a devastating consequence of cytotoxic chemotherapy and radiotherapy that drastically affects the quality of life of people undergoing such treatment. In recent years a class of drugs referred to as 5-HT₃ (5-hydroxytryptamine) receptor antagonists has been developed that treat such emesis by antagonizing cerebral functions associated with the 5-HT₃ receptor. See Drugs Acting on 5-Hydroxytryptamine Receptors: The Lancet Sep. 23, 1989 and refs. cited therein. Drugs within this class include ondansetron, granisetron and dolasetron, as disclosed in U.S. Pat. Nos. 4,695,578, 4,753,789, 4,929,632, 5,240,954, 5,578,628, 5,578,632, 5,922,749, 5,622,720, 5,955,488, and 6,063,802 (ondansetron), U.S. Pat. Nos. 4,906,755, 5,011,846, and 4,906,755 (dolasetron), and U.S. Pat. Nos. 4,886,808, 4,937,247, 5,034,398 and 6,294,548 (granisetron).

These 5-HT₃ antagonists are typically administered intravenously shortly before chemotherapy or radiotherapy is initiated, and can be administered more than once during a cycle of chemotherapy or radiotherapy. For example, when a chemotherapeutic agent is administered more than once in a cycle (as for example on days 1-7 in a 28 day chemotherapy cycle), the 5-HT₃ antagonist can be administered on each of days 1-7 for optimum anti-emetogenic effect. Because some chemotherapeutic agents can induce emesis over extended periods of several days even when they are administered only once, it would be desirable to administer an emesis-inhibiting drug such as a 5-HT₃ antagonist every day until the risk of emesis has substantially subsided. The present class of 5-HT₃ antagonists has not proven especially helpful meeting this need, however, because the 5-HT₃ receptor antagonists currently marketed have proven to be less effective in controlling delayed nausea and vomiting than they are at controlling acute emesis. Sabra, K, Choice of a SHT₃ Receptor Antagonist for the Hospital Formulary. EHP, October 1996;2 (suppl 1):S19-24.

Each of the currently available 5-HT₃ antagonists also suffers from one or more of the following deficiencies which limits its therapeutic utility: potency, duration of effect, window of therapeutic efficacy, ease of dosing, side effects, and certainty of the dosing regimen. Sabra, K (1996) (supra). Although side effects are typically mild to moderate and transient, they include headache, lightheadedness or dizziness, abdominal pain or cramping, constipation, sedation and fatigue, elevations in hepatic transaminases and/or bilirubin, and electrocardiographic changes. Gregory, R E and Ettinger, D S, 5HT₃ receptor antagonists for the prevention of chemotherapy-induced nausea and vomiting. A comparison of their pharmacology and clinical efficacy. Drugs, February 1998;55(2):173-189.

Various patents and references disclose classes of compounds useful as emesis-inhibiting agents and 5-HT₃ antagonists. For example, Ponchant et al., “Synthesis of 5-¹²⁵I-Iodo-Zacopride, A New Probe for 5-HT₃ Receptor Sites,” J. Lab. Cpds. and Radiopharm., Vol. XXIX, No. 10, pp. 1147-1155 (1991) discloses substituted 3-quinuclidinyl benzamides useful for 5-HT₃ serotonin receptor binding. U.S. Pat. No.4,717,563 to Alphin et al., discloses a method of controlling emesis caused by non-platinum anti-cancer drugs utilizing particular N-3-quinuclidinyl benzamides and thiobenzamides. U.S. Pat. No. 4,820,715 to Monkovic et al. discloses substituted 3-quinuclidinyl benzamide compounds which are asserted to be useful for the treatment of emesis, such as chemotherapy-induced emesis, and/or treatment of disorders related to impaired gastric motility. U.S. Pat. No. 5,202,303 to Berger et al. discloses a class of benz[de]isoquinolin-1-ones that act as 5-HT₃ receptor antagonists. Species disclosed in the patent include palonosetron. According to this patent, the class of compounds is useful to treat emesis, gastrointestinal disorders, anxiety, depressive states, and pain. The patent does not disclose any particular data for determining a suitable therapeutic regimen such as the potency of the compounds, the serum half life of the compounds, dose response data, or duration of effect.

One of the greatest challenges in drug dosing, especially when multiple doses are administered over a period of a few days, is to find a dose that is well-tolerated and consistently efficacious throughout the dosing regimen. Finding an optimum dose is complicated by such factors as serum half-life, dosing/efficacy relationships, and, in the case of anti-emetic drugs, the variability of the chemotherapeutic regimen with which the drugs will be administered and the types of emesis induced (i.e. delayed v. acute emesis and moderately v. highly emetogenic chemotherapy). This challenge is particularly acute when devising single unit dose formulations of the anti-emetic drug that is efficacious over a range of body weights, because single unit dose forms are designed typically to prevent nurses and doctors from titrating the dose in the clinic.

### OBJECTS OF THE INVENTION

It is an object of the present invention to provide methods of inhibiting emesis using 5-HT₃ receptor antagonists that have heightened potency, and that can be administered in lower doses to yield fewer incidences of unwanted side effects.

It is another object of the invention to provide methods of inhibiting and relieving acute and delayed emesis induced by chemotherapeutic agents or radiotherapy, and drugs having the capacity to inhibit and relieve such acute and delayed emesis.

Another object of the present invention is to provide a uniform defined dose of palonosetron that can be administered to a patient of nearly any body weight in successive 24 hour periods to control immediate and delayed emesis.

Still another object of the present invention is to provide 5-HT₃ antagonists that possess increased plasma half-life and prolonged in vivo activity.

Another object of the invention is to provide greater flexibility when administering emesis-inhibiting agents in advance of chemotherapeutic regimens or radiotherapy, by increasing the size of the window for pretreatment.

Yet another object of the invention is to provide greater flexibility when administering an emesis-inhibiting agent, by reducing the time required to administer a bolus of the emesis-inhibiting agent Still another object of the present invention is to provide more definite regimens for the treatment of chemotherapeutically or radiotherapeutically induced emesis, by preventing doctors from prescribing unnecessarily increased dosages of palonosetron, and allowing doctors to switch to other anti-emetic agents when palonosetron does not prove effective rather than increasing the dose.

### SUMMARY OF INVENTION

Contrary to published reports that 5-HT₃ antagonists have minimal benefit in the treatment or prevention of delayed emesis, the inventors have surprisingly discovered that palonosetron alleviates and prevents acute and delayed emesis in both moderately and highly emetogenic chemotherapy regimens, and that palonosetron is substantially superior to both ondansetron and dolasetron in its ability to prevent and alleviate nausea and vomiting that occurs more than 24 hours after the initiation of moderately emetogenic chemotherapy. Therefore, in one embodiment the invention provides a method of treating acute and delayed emesis in a patient undergoing emetogenic chemotherapy or radiotherapy comprising administering a treatment effective amount of palonosetron. The treatment effective amount is preferably one of the doses disclosed in greater detail elsewhere in this document.

The inventors have also made a series of discoveries that support a surprisingly effective and versatile clinical regimen for the treatment and prevention of emesis using palonosetron. In particular, the inventors have discovered:
that palonosetron can treat and prevent emesis induced by chemotherapy and radiotherapy at levels of only about 1/10^{th} the levels of other 5-HT₃ antagonists;
that palonosetron has a plasma half-life of about 40 hours; and
that the efficacy of palonosetron plateaus over a broad range of doses ranging from about 30 μg/kg to about 90 μg/kg.

Based upon the foregoing discoveries, the inventors have determined that 0.25 mg/day of palonosetron is a particularly effective and versatile dose of palonosetron for use in the clinic because the dose is effective when used only once in a chemotherapy or radiotherapy cycle, but it is also effective when administered on successive days because, in spite of the long half-life of palonosetron and a concomitant build-up of palonosetron during the multiple doses, consistent efficacy can be expected from each dose of palonosetron due to the observed plateau effect. Moreover, such efficacy can be expected over a wide range of patient body weights.

Therefore in another embodiment the invention provides a method of treating chemotherapy or radiotherapy-induced emesis in a patient comprising administering a dose of 0.25 mg of palonosetron to the patient. In another embodiment the invention provides single unit dosage forms of palonosetron that comprise 0.25 mg of palonosetron.

The surprising potency of palonosetron, the extended plasma half-life of palonosetron, and the plateau dosing phenomena observed for palonosetron, also have a number of other practical advantages including:
The potency of palonosetron allows for greater cost efficiencies because lesser quantities of palonosetron are needed.
The potency of palonosetron also allows the manufacturer to formulate the drug at reduced concentrations. This advantage is of particular significance in the formulation of palonosetron because palonosetron has been found to be most stable at lower concentrations. This advantage is also significant from a convenience standpoint because the palonosetron can be sufficiently concentrated to be intravenously administered as a bolus in only about from 10 to 30 seconds.
The extended plasma half-life reduces even further the quantity of palonosetron that must be administered to treat emesis.
The extended half-life allows the palonosetron to be administered at intervals of about 24 hours, or only once in certain therapeutic settings.
The extended half-life allows greater flexibility in a clinical setting by allowing the drug to be administered over a larger window of time preceding the administration of chemotherapy or radiotherapy.
The extended half-life and plateau dosing phenomena combine to provide more certainty to the regimen by assuring the physician that a further dose of palonosetron in a session is not warranted within a particular window of time even if emesis is experienced.
The extended half-life and plateau dosing phenomena further combine to provide more certainty to the regimen by avoiding the tendency of physicians to increase the dose of palonosetron in subsequent sessions if emesis is experienced in a prior session.

### DEFINITIONS

“Ampule” means a small sealed container of medication that is used one time only, and includes breakable and non-breakable glass ampules, breakable plastic ampules, miniature screw-top jars, and any other type of container of a size capable of holding only one unit dose of palonosetron (typically about 5 mls.).

“Animal” includes humans, non-human mammals (e.g., dogs, cats, rabbits, cattle, horses, sheep, goats, swine, and deer) and non-mammals (e.g., birds and the like). While the methods disclosed herein are generally applicable to people, they are applicable as well to animals.

“Chemotherapeutic agents” include a variety of classes of compounds for treating proliferative disorders including alkylating agents, antimetabolites, natural products, enzymes, biological response modifiers, miscellaneous agents, radiopharmaceuticals (for example, Y-90 tagged to hormones or antibodies), hormones and antagonists, such as those listed below.

Antiangiogenesis Agents: Angiostatin, Endostatin.

Alkylating Agents: Nitrogen Mustards such as Mechlorethamine, Cyclophosphamide, Ifosfamide, Melphalan (L-sarcolysin),and Chlorambucil; Ethylenimines and Methylmelamines such as Hexaamethylmelamine and Thiotepa; Alkyl Sulfonates such as Busulfan; Nitrosoureas such as Carmustine (BCNU), Lomustine (CCNU), Semustine (methyl-CCNU), and Streptozocin (STR); and Triazenes such as Dacarbazine (DTIC; dimethyltriazenoimidazole-carboxamide).

Antimetabolites: Folic Acid Analogs such as Methotrexate (amethopterin); Pyrimidine Analogs such as Fluorouracil (5-fluorouracil; 5-FU), Floxuridine (fluorodeoxyuridine; FUdR), and Cytarabine (cytosine arabinoside); Purine Analogs and Related Inhibitors such as Mercaptopurine (6-mercaptopurine; 6-MP), Thioguanine (6-thioguanine: TG), and Pentostatin (2′-deoxycyoformycin); Vinca Alkaloids such as Vinblastine (VLB), and Vincristine; and Epipodophylotoxins such as Etoposide and Teniposide.

Natural Products: Antibiotics such as Dactinomycin (actinonmycin D), Daunorubicin (daunomycin; rubidomycin), Doxorubicin, Bleomycin, Plicamycin (mithramycin), and Mitomycin (mitomycin C); Enzymes such as L-Asparaginase; and Biological Response Modifiers such as Interferon-alfa.

Miscellaneous Agents: Platinum Coordination Complexes such as Cisplatin (cis-DDP) and Carboplatin; Mixtozantrone; Hydroxyurea; Procarbazine (N-methylhydrazine, MIH); Mitotane (o,p′-DDD); Aminoglutethimide; Adrenorticosteriods such as Prednisone; and Progestins such as Hydroxprogesterone caproate, Medroxyprogesterone acetate, and Megestrol acetate.

Hormones and Antagonists: Estrogens such as Diethylstibestrol and Ethinyl estradiol; Antiestrogens such as Tamoxifen; Androgens such as Testosterone propionate Fluxomyesterone; Antiandrogens such as Flutamide (prostate); and Gonadotropin-Releasing Hormone Analog such as Leuprolide.

Throughout this specification the word “comprise,” or variations such as “comprises” or “comprising,” will be understood to imply the inclusion of a stated element, integer or step, or group of elements, integers or steps, but not the exclusion of any other element, integer or step, or group of elements, integers or steps

“Disease” specifically includes any unhealthy condition of an animal or part thereof and includes an unhealthy condition which may be caused by, or incident to, medical or veterinary therapy applied to that animal, i.e., the side effects of such therapy. Thus, disease here includes the emesis caused by therapy with agents having emetogenic side effects, in particular by therapy for cancer, such as chemotherapy with chemotherapeutic agents and radiotherapy.

“Emesis”, for the purposes of this application, will have a meaning that is broader than the normal, dictionary definition and includes not only vomiting, but also nausea and retching.

“Delayed emesis” means emesis that occurs greater than about 24 hours after initiation of an emesis inducing chemotherapeutic or radiotherapeutic event. Delayed emesis thus includes emesis that occurs up to 2, 3, 4, or even 5 days after a chemotherapeutic or radiotherapeutic event.

“Acute emesis” refers to emesis that involves vomiting within 24 hours of initiation of an emesis inducing chemotherapeutic or radiotherapeutic event.

“Moderately emetogenic chemotherapy” refers to chemotherapy in which the emetogenic potential is comparable or equivalent to the emetogenic potential of carboplatin, cisplatin ≦50 mg/m², cyclophosphamide <1500 mg/m², doxorubicin >25 mg/ms, epirubicin, irinotecan, or methotrexate >250 mg/m².

“Highly emetogenic chemotherapy” refers to chemotherapy in which the emetogenic potential is comparable or equivalent to the emetogenic potential of cisplatin ≧60 mg/m², cyclophosphamide >1500 mg/m², or dacarbazine.

“Palonosetron” means (3aS)-2,3,3a,4,5,6-Hexahydro-2-[(S)-1-Azabicyclo[2.2.2]oct-3-yl]2,3 ,3a,4,5,6-hexahydro-1-oxo-1Hbenz[de]isoquinoline hydrochloride, and is preferably present as the monohydrochloride. Palonosetron monohydrochloride can be represented by the following chemical structure:

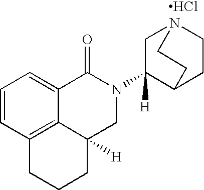

“Pharmaceutically acceptable” means that which is useful in preparing a pharmaceutical composition that is generally safe, non-toxic and neither biologically nor otherwise undesirable and includes that which is acceptable for veterinary use as well as human pharmaceutical use.

“Pharmaceutically acceptable salts” means salts which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as acetic acid, propionic acid, hexanoic acid, heptanoic acid, cyclopentanepropionic acid, glycolic acid, pyruvic acid, lactic acid, malonic acid, succinic acid, malic acid, maleic acid, fumaric acid, tartaric acid, citric acid, benzoic acid, o-(4-hydroxybenzoyl)benzoic acid, cinnamic acid, mandelic acid, methanesulfonic acid, ethanesulfonic acid, 1,2,-ethanedisulfonic acid, 2-hydroxyethanesulfonic acid, benzenesulfonic acid p-chlorobenzenesulfonic acid, 2-naphthalenesulfonic acid, p-toluenesulfonic acid, camphorsulfonic acid, 4-methylbicyclo[2.2.2]oct-2-ene-1-carboxylic acid, glucoheptonic acid, 4,4′-methylenebis(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like.

In addition, pharmaceutically acceptable salts may be formed when an acidic proton present is capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like.

“Therapeutically effective amount” means that amount which, when administered to an animal for treating a disease, is sufficient to effect such treatment for the disease.

“Treating” or “treatment” of a disease includes: (1) preventing the disease from occurring in an animal which may be predisposed to the disease but does not yet experience or display symptoms of the disease, (2) treating the disease, i.e., arresting its development, or (3) relieving the disease, i.e., causing regression of the disease.

### DETAILED DISCUSSION

One embodiment of the present invention is premised upon the discovery that palonosetron is surprisingly superior to other 5-HT₃ antagonists in its ability to treat delayed emesis, while exhibiting remarkable efficiency at treating acute emesis as well. Thus, in one embodiment, the invention provides a method of treating chemotherapy or radiotherapy induced delayed and acute emesis comprising administering a treatment-effective amount of palonosetron. The method is effective for the treatment of emesis induced by both moderately emetogenic chemotherapy and highly emetogenic chemotherapy.

In another embodiment the invention provides a method for treating acute and/or delayed emesis comprising (a) administering to a human or other animal from about 1 to about 10 ug/kg of palonosetron, and (b) administering to said human or other animal an emesis inducing amount of a chemotherapeutic agent or radiotherapy. While larger doses of palonosetron of up to about 30, 60, or 90 ug/kg, can be administered effectively to reduce emesis, it has surprisingly been found that the effectiveness of palonosetron typically plateaus at these lower concentrations in the chemotherapeutic regimens tested. Therefore, in a preferred embodiment, from about 3 to about 10 ug/kg of palonosetron is administered. In another embodiment the palonosetron is administered in the absence of a steroid such as dexamethasone.

In another embodiment the invention provides a method of treating chemotherapy or radiotherapy-induced emesis in a patient comprising administering a dose of 0.25 mg of palonosetron to the patient. This dose has been found to be effective over a range of body weights from about 40 to about 120 kilograms , and can conveniently be provided in single unit dose ampules that need not be titrated based upon a patient's body weight within this range. The dose is effective to treat chemotherapy-induced delayed and acute emesis. In addition, the dose is effective for the treatment of emesis induced by both moderately emetogenic chemotherapy and highly emetogenic chemotherapy.

A particularly surprising advantage of the lower dosages required of palonosetron derives from the fact that the stability of palonosetron increases in solution as its concentration decreases. The potency of palonosetron thus allows the palonosetron to be formulated in stable compositions comprising a wide range of palonosetron concentrations, preferably from about 0.01 mg/ml to about 0.20 mg/ml palonosetron, most preferably at a concentration of about 0.05 mg/ml. Thus, in one particular embodiment the palonosetron is supplied in ampules that comprise 5 ml. of solution, which equates to about 0.25 mg of palonosetron at a concentration of about 0.05 mg/ml.

The enhanced stability allows the palonosetron to be stored for extended periods of time, exceeding about 1 month, 3 months, 6 months, 1 year, or 18 months, but preferably not extending beyond 30 months (we are testing the stability, which shall be included in the FDA file). This enhanced stability is seen in a variety of storage conditions, including room temperature.

The method can be practiced using virtually any method of administration including oral, systemic (e.g., transdermal, intranasal or by suppository) or parenteral (e.g., intramuscular, intravenous or subcutaneous). In a preferred embodiment the palonosetron is administered as an oral liquid or intravenously, and most preferably the palonosetron is administered intravenously.

Another particular advantage associated with the lower dosages of palonosetron is the ability to administer the drug in a single intravenous bolus over a short, discrete time period. This time period generally extends from about 10 to about 60 seconds, or about 10 to about 40 seconds, and most preferably is about 10 to 30 seconds.

Still other embodiments pertain to the sequence and timing of steps in which the method is performed. Although the method can be performed in any sequence, in a preferred embodiment step (a) (the palonosetron administration) precedes step (b) (the chemotherapy or radiotherapy administration) in sequence. Moreover, while step (a) can be performed over a large window of time preceding step (b), from immediately before step (b) to as long as 1, 2, 5, 8, or 10 hours before step (b), the palonosetron is preferably administered from about 15 minutes to about 2 hours before the chemotherapeutic agent or radiotherapy, more preferably from about 15 minutes to about 1 hour before the chemotherapeutic agent or radiotherapy, and most preferably about 30 minutes before the chemotherapy agent or radiotherapy.

The methods of the present invention are preferably performed in the context of chemotherapeutic or radiotherapy cycles, in which an intensive regimen of chemotherapy or radiotherapy is administered over a defined time period, followed by an extended recovery period, and a subsequent cycle in which the therapy and recovery sequence is repeated. An intensive regimen of chemotherapy may comprise only one administration of a chemotherapeutic agent, or it may comprise several days of administering the same chemotherapeutic agent. Similarly, the regimen may include the administration of more than one chemotherapeutic agents. Moreover, one or more of the chemotherapeutic agents may induce acute and/or delayed emesis.

Palonosetron is preferably administered in conjunction with the chemotherapy or radiotherapy based upon when emesis is most likely to be experienced, at time intervals of about 24 hours. For example, if an emesis inducing chemotherapeutic agent that induces acute emesis and not delayed emesis is administered only once during a cycle of chemotherapy (i.e. in one session), palonosetron will be administered only once as well. If the chemotherapeutic agent induces acute and delayed emesis, then palonosetron will preferably be administered at the initiation of the chemotherapy and every 24 hours thereafter until the emesis has subsided. Similarly, if an emesis inducing agent is administered more than once during a chemotherapy cycle (i.e. in more than one session), then palonosetron will preferably also be administered more than once every 24 hours.

The methods of the present invention are useful for treating emesis induced by a wide range of chemotherapeutic agents and radiotherapies discussed above, but are particularly useful when employed in conjunction with cisplatin, cyclophosphamide, carmustine, dicarbazine, actinomycin D, mechlorethamine, carboplatin, doxorubicin, epirubicin, irinotecan, methotrexate, and dacarbazine. The methods have also been found to be particularly useful when used in conjunction with highly emetogenic chemotherapy, especially when using chemotherapeutic agents that are typically associated with extended periods of emesis, or delayed onset of emesis. In various embodiments, the chemotherapeutic agent induces emesis for a period of at least about 24 hours, 48 hours, 72 hours, or 5 days. For example, in one embodiment the chemotherapeutic agent is cisplatin or cyclophosphamide, and in still further preferred embodiments the cisplatin is administered at a dose exceeding about 30, 40, 50, 60, or 70 mg/m², and the cyclophosphamide is administered at a dose exceeding about 500, 600, 700, 800, 900, 1000, or 1100 mg/m².

The plateau effects observed with increasing doses of palonosetron, combined with its extended half-life, further combine to inform the prescribing physician of appropriate regimens to be followed when a patient is at least partially non-responsive to palonosetron. For example, under some circumstances the physician will be assured that he should switch the patient from palonosetron in a subsequent session rather than administer a higher dose of palonosetron. Therefore, in another embodiment the invention provides a method of preventing chemotherapy induced emesis comprising: (1) in a first chemotherapeutic session: administering to a human or other animal a first amount of palonosetron; and administering to said human or other animal an emesis inducing amount of a chemotherapeutic agent; (2) assessing the effectiveness of the palonosetron; and (3) in a subsequent chemotherapeutic session, if said human or other animal is at least partly non-responsive to said palonosetron in said first chemotherapeutic session, administering to said human or other animal a therapeutically effective amount of a second anti-emetic compound; wherein the subsequent chemotherapeutic session is performed without an intervening chemotherapeutic session in which a second amount of palonosetron higher than the first amount is administered.

The plateau dosing effect, when combined with the extended serum half life, can also render the dosing regimen in some circumstances more certain by avoiding the tendency of physicians to prematurely administer additional doses of palonosetron in a single therapeutic session if emesis is experienced. Thus, in another embodiment the invention provides a method of preventing chemotherapy induced emesis comprising, (1) in one chemotherapeutic session: administering to a human or other animal a first dose of palonosetron; and administering to said human or other animal an emesis inducing amount of a chemotherapeutic agent; (2) assessing the effectiveness of the palonosetron; and (3) if said human or other animal is at least partly non-responsive to said first dose of palonosetron, not administering a second dose of palonosetron for at least about 20, 24, 28, or 32 hours after step (a) or (b).

### Injection Administration

In a preferred embodiment the palonosetron is formulated in an injectable solution and subsequently mixed with an infusion solution selected from dextrose injection (most commonly 5%), NaCl 0.9% injection (i.e. normal saline), lactated Ringer's solution, Ringer's solution, and water. The palonosetron will typically have a concentration after mixing with any of these infusion solutions of from about 1 to about 100 mcg/mL, more preferably from about 2 to about 50 mcg./mL, and most preferably from about 5 to about 30 mcg./mL.

Thus, for example, suitable infusion solutions with which the palonosetron can be mixed include:

Dextrose 5% in Water (D5W)

Dextrose 5% in Lactated Ringers (D5LR)

Dextrose 5% in Normal Saline (D5S)

Dextrose 5% in 1/2 (0.45%) strength Saline (D5-1/2S)

Dextrose 5% in 1/4 (0.225%) strength Saline (D5-1/4S)

Dextrose 10% in Water (D10W)

Dextrose 20% in Water (D20W)

Dextrose 50% in Water (D50W)

2/3(3.33%)Dextrose 5% & 1/3(0.3%)Saline 0.9% (2/3D,1/3S)

Lactated Ringers Solution (LR)

Ringers Solution (RS)

Sodium Chloride 0.9% (Normal Saline) NS

The palonosetron can be stored in any of the above infusion solutions, typically in a plastic bag made from polyvinyl chloride, for two days and even more if refrigerated.

The palonosetron will typically be administered using conventional Y-site administration in combination with a chemotherapeutic agent known to induce emesis, selected from the group discussed above in greater detail. Y-site administration is an infusion process in which an external line is provided at the Y-juncture for addition of other drugs such as palonosetron during a chemotherapeutic regimen. The administration can be either sequential or simultaneous.

### Pharmaceuticals Compositions

Although the foregoing discussion has focused upon intravenous administration of palonosetron because that is the preferred mode of administration, the methods of the present invention can be performed by administering palonosetron by any one of the following routes: oral, systemic (e.g., transdermal, intranasal or by suppository) or parenteral (e.g., intramuscular, intravenous or subcutaneous).

Oral compositions will generally include an inert diluent or an edible carrier. They may be enclosed in gelatin capsules or compressed into tablets. For the purpose of oral therapeutic administration, the active compound can be incorporated with excipients and used in the form of tablets, troches, or capsules. Pharmaceutically compatible binding agents, and/or adjuvant materials can be included as part of the composition.

The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate; a glidant such as colloidal silicon dioxide; a sweetening agent such as sucrose or saccharin; or a flavoring agent such as peppermint, methyl salicylate, or orange flavoring. When the dosage unit form is a capsule, it can contain, in addition to material of the above type, a liquid carrier such as a fatty oil. In addition, dosage unit forms can contain various other materials which modify the physical form of the dosage unit, for example, coatings of sugar, shellac, or other enteric agents.

The compound can be administered as a component of an elixir, suspension, syrup, wafer, chewing gum or the like. A syrup may contain, in addition to the active compounds, sucrose as a sweetening agent and certain preservatives, dyes and colorings and flavors.

The compound or a pharmaceutically acceptable prodrug or salts thereof can also be mixed with other active materials that do not impair the desired action, or with materials that supplement the desired action, such as antibiotics, antifungals, anti-inflammatories, or other antivirals, including other nucleoside compounds. Solutions or suspensions used for parenteral, intradermal, subcutaneous, or topical application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parental preparation can be enclosed in ampoules, disposable syringes or multiple dose vials made of glass or plastic.

If administered intravenously, preferred carriers are physiological saline or phosphate buffered saline (PBS).

Preferably the pharmaceutical composition is administered in a single unit dosage form for continuous treatment or in a single unit dosage form ad libitum when relief of symptoms is specifically required.

### EXAMPLES

### Example 1

### Antiemetic Effects of Palonosetron in Animal Models

The antiemetic effects of intravenously and orally administered palonosetron were assessed in male and female dogs (sexes combined). Antineoplastic agents employed in these experiments included cisplatin (3 mg/kg) ), dacarbazine (30 mg/kg), mechlorethamine (0.4 mg/kg) and actinomycin D (0.15 mg/kg).

Palonosetron and comparator drugs were administered prior to or after antineoplastic agents and animals were observed for the number of emetic episodes. Mean numbers of episodes were computed and the statistical significance of differences between treatment groups were determined with Dunnett's t test. Solutions of palonosetron and comparator drugs (ondansetron and granisetron) were prepared in distilled water. Vehicle control groups received distilled water.

The therapeutic effects of intravenously and orally administered palonosetron were compared to those of ondansetron and vehicle in cisplatin-treated dogs. When administered by the intravenous route, both palonosetron (0.1 mg/kg) and ondansetron (1.0 mg/kg) reduced the number of emetic episodes during the five-hour period following administration of cisplatin 3.0 mg/kg. Palonosetron was statistically significantly more potent than ondanestron based on the mean number of emetic episodes observed (2.20 and 6.83 for palonosetron and ondansetron, respectively, compared with 12.50 for vehicle control).

In a different study, palonosetron and ondansetron were assessed for their relative abilities to reverse cisplatin-induced emesis. In these experiments dogs received intravenous injections of palonosetron, ondansetron, or vehicle one hour after the intravenous administration of 3.0 mg/kg cisplatin. Each animal was observed continuously for five hours following cisplatin administration for the number of emetic episodes. The results of this experiment are summarized in Table 1.

TABLE 1
Therapeutic Effect of Intravenously Administered Palonosetron and
Ondansetron on Cisplatin-Induced Emesis in Dogs

| | | Intravenous Dose | Emetic Episodes |
|---|---|---|---|
| Treatment | N | (μg/kg) | (mean ± SD) |
| Vehicle control | 6 | 0.0 | 13.50 ± 5.89 |
| Palonosetron | 6 | 0.3 | 13.16 ± 6.11 |
| | 6 | 1.0 | 7.16 ± 4.26^{a} |
| | 6 | 3.0 | 4.33 ± 4.03^{b} |
| | 6 | 10.0 | 4.50 ± 5.24^{b} |
| | 6 | 30.0 | 1.33 ± 1.21^{b} |
| | 6 | 100.0 | 1.67 ± 1.83^{b} |
| | 6 | 300.0 | 3.33 ± 3.01^{b} |
| Ondansetron | 6 | 1.0 | 16.16 ± 5.14 |
| | 6 | 3.0 | 13.33 ± 4.17 |
| | 6 | 10.0 | 13.33 ± 3.01 |
| | 6 | 30.0 | 8.33 ± 3.20 |
| | 6 | 100.0 | 4.83 ± 2.40^{b} |
| | 6 | 300.0 | 1.00 ± 2.00^{b} |
| | 6 | 1000.0 | 1.16 ± 0.40^{b} |

^{a}Significantly less than vehicle control, p < 0.05.
^{b}Significantly less than vehicle control, p < 0.01.

The statistically minimally effective dose level was 1.0 μg/kg for palonosetron and 100 μg/kg for ondansetron, suggesting that palonosetron was substantially more potent than ondansetron in this experiment.

The observation of potency differential was further seen during an experiment in which oral dose-response relationships of palonosetron and ondansetron were compared in cisplatin-treated dogs. Palonosetron, ondansetron or vehicle control were administered by the oral route one hour prior to the administration of cisplatin. Each animal was observed continuously for five hours following cisplatin administration for the number of emetic episodes. The results of this experiment are summarized in Table 2.

TABLE 2
Anti-Emetic Effect in Male and Female Dogs of Palonosetron or
Ondansetron Orally Administered One Hour Before Cisplatin

| | | | Emetic Episodes |
|---|---|---|---|
| Treatment | N | Oral Dose (μg/kg) | (mean ± SD) |
| Vehicle control | 6 | 0.0 | 14.16 ± 7.98 |
| Palonosetron | 6 | 0.3 | 12.16 ± 1.94 |
| | 6 | 1.0 | 12.83 ± 4.70 |
| | 6 | 3.0 | 16.16 ± 5.11 |
| | 6 | 10.0 | 4.00 ± 3.79^{a} |
| | 6 | 30.0 | 2.66 ± 1.75^{a} |
| | 6 | 100.0 | 0.00 ± 0.00^{a} |
| | 6 | 300.0 | 1.67 ± 2.04^{a} |
| Ondansetron | 6 | 1.0 | 15.50 ± 3.93 |
| | 6 | 3.0 | 14.83 ± 6.49 |
| | 6 | 10.0 | 12.66 ± 3.01 |
| | 6 | 30.0 | 18.33 ± 8.43 |
| | 6 | 100.0 | 10.50 ± 3.14 |
| | 6 | 300.0 | 3.16 ± 1.32^{a} |
| | 6 | 1000.0 | 2.00 ± 2.09^{a} |

^{a}Significantly less than vehicle control, p < 0.01.

Pretreatment with both palonosetron and ondansetron reduced the incidences of emetic episodes. Palonosetron exhibited the greater potency as evidenced by a significant reduction in episodes after 10 μg/kg as compared to the minimally effective dose of 300 μg/kg for ondansetron.

### Example 2

### Duration of Action in Cisplatin-Treated Dogs

The durations of action of intravenously administered palonosetron, ondansetron and granisetron in dogs were compared in an experiment, the results of which are shown in Table 3. In this study groups of six dogs received intravenous doses of palonosetron, ondansetron, or granisetron (0.1, 0.15, or 0.04 mg/kg, respectively), or vehicle control 12, 10, 8, 6, 4, 2, or 1 hour prior to the intravenous injection of 3.0 mg/kg of cisplatin.

TABLE 3
Duration of Action of Intravenously Administered Palonosetron,
Ondansetron, or Granisetron in Cisplatin-Dosed Dogs

| | | Dose | |
|---|---|---|---|
| Pre-Cisplatin (hr) | Treatment | (mg/kg) | Emetic Episodes (mean ± SD) |
| 12 | Vehicle | 0 | 17.33 ± 4.68 |
| | Palonosetron | 0.1 | 13.17 ± 6.27 |
| | Ondansetron | 0.15 | 22.00 ± 8.32 |
| | Granisetron | 0.04 | 15.67 ± 6.28 |
| 10 | Vehicle | 0 | 19.33 ± 5.68 |
| | Palonosetron | 0.1 | 9.83 ± 4.79^{b} |
| | Ondansetron | 0.15 | 13.83 ± 4.58 |
| | Granisetron | 0.04 | 23.00 ± 5.87 |
| 8 | Vehicle | 0 | 20.00 ± 4.94 |
| | Palonosetron | 0.1 | 9.50 ± 5.50^{b} |
| | Ondansetron | 0.15 | 15.50 ± 7.29 |
| | Granisetron | 0.04 | 14.67 ± 8.69 |
| 6 | Vehicle | 0 | 17.33 ± 7.66 |
| | Palonosetron | 0.1 | 5.83 ± 4.40^{b} |
| | Ondansetron | 0.15 | 14.17 ± 6.79 |
| | Granisetron | 0.04 | 21.33 ± 9.54 |
| 4 | Vehicle | 0 | 12.67 ± 2.34 |
| | Palonosetron | 0.1 | 0.33 ± 0.52^{b} |
| | Ondansetron | 0.15 | 12.67 ± 2.07 |
| | Granisetron | 0.04 | 13.83 ± 7.25 |
| 2 | Vehicle | 0 | 12.00 ± 6.20 |
| | Palonosetron | 0.1 | 2.50 ± 2.43^{b} |
| | Ondansetron | 0.15 | 10.67 ± 5.28 |
| | Granisetron | 0.04 | 9.67 ± 4.46 |
| 1 | Vehicle | 0 | 13.83 ± 4.36 |
| | Palonosetron | 0.1 | 1.33 −+ 1.03^{b} |
| | Ondansetron | 0.15 | 7.83 ± 4.12^{b} |
| | Granisetron | 0.04 | 12.33 ± 4.32 |

^{a}For each treatment group, n = 6 and the vehicle control was distilled water.
^{b}Significantly less than vehicle control, p < 0.05 using Fisher's LSD strategy.

Palonosetron exhibited some antiemetic activity when administered as long as 10 hours before the injection of cisplatin. Ondansetron reduced emetic episodes only after a one-hour pretreatment period. Granisetron was without antiemetic effect in these experiments. Palonosetron did not protect when administered 12 hours before cisplatin. In an earlier experiment, a higher dose of ondansetron (0.3 mg/kg, IV) exhibited protective effects when administered as long as seven hours prior to injection of cisplatin. In that experiment, a dose of 0.03 mg/kg of palonosetron was similarly effective. The results of these experiments support the conclusion that the duration of antiemetic action of palonosetron is longer than that of ondansetron, and that a higher dose of ondansetron is required to achieve an equivalent effect.

### Example 3

### Relationship Between Systemic Exposure and Antiemetic Effect

An experiment was conducted to study the relationship between plasma concentration of palonosetron and protection of dogs against cisplatin-induced emesis. Groups of dogs received oral doses of palonosetron (0, 100, 316, or 1000 μg/kg) or vehicle control 30 minutes prior to the injection of cisplatin. Plasma concentrations of palonosetron were determined by an HPLC-radioimmunoassay method at 0, 0.25, 0.5, 1, 2, 4, 8, 24, and 48 hours after administration of palonosetron and systemic exposure was expressed as AUCO-4 hr. Results are summarized in Table 4.

TABLE 4
Systemic Exposure of Palonosetron (AUC_{0-4 hr}) in Dogs After Intravenous
Administration and Effects on Cisplatin-Induced Emesis

| | | | Emetic Episodes |
|---|---|---|---|
| Palonosetron (μg/kg) | N | AUC_{0-4 hr} (mean ± SD) | (mean ± SD) |
| 0 (vehicle) | 6 | 0.00 ± 0.00^{a} | 15.83 ± 3.43 |
| 100 | 6 | 10.59 ± 7.24^{a} | 1.83 ± 1.60^{b} |
| 316 | 6 | 30.64 ± 12.02^{a} | 4.50 ± 6.83^{b} |
| 1000 | 6 | 76.07 ± 50.39^{a} | 5.67 ± 5.32^{b} |

^{a}Significantly different from all other AUCs, p < 0.01.
^{b}Significantly different from controls, p < 0.05.

Systemic exposure to palonosetron, as estimated by computed AUC_{0-4 hr} values, were approximately dose-proportional over the range studied, however, a relationship between systemic exposure and magnitude of antiemetic effect could not be demonstrated. Dogs dosed with palonosetron had significantly fewer emetic episodes than vehicle control animals but there was no evidence of a significant dose-response relationship. The lowest dose tested appeared to be at the response plateau.

### Example 4

### Antagonism of Emetic Effects of Dacarbazine, Actinomycin D, and Mechlorethamine

The protective effects of palonosetron and ondansetron against emesis caused by the administration of dacarbazine, actinomycin D, or mechlorethamine were assessed in dogs. In these experiments palonosetron, ondansetron or vehicle were administered 2 hours prior to the injection of the antineoplastic agent and animals were observed for 5 hours. The results of these experiments are summarized in Table 5.

TABLE 5
Effects of Palonsetron and Ondansetron on Dacarbazine-, Actinomycin D-,
and Mechlorethamine-Induced Emesis in Dogs

| | | Emetic Episodes (mean ± SD) | | |
|---|---|---|---|---|
| | | Dacarbazine | Actinomycin D | Mechlorethamine |
| Treatment^{a} | Dose (mg/kg) | (30.0 mg/kg IV) | (0.15 mg/kg IV) | (0.4 mg/kg IV) |

Intravenous Antiemetic

| | | | | |
|---|---|---|---|---|
| Vehicle control^{b} | 0 | 10.33 ± 2.66 | 17.50 ± 11.43 | 10.00 ± 4.69 |
| Palonosetron | 0.001 | 10.17 ± 2.04 | 10.00 ± 0.54^{c} | 7.67 ± 5.05 |
| | 0.003 | 6.67 ± 4.13 | 10.83 ± 2.04 | 6.00 ± 4.29 |
| | 0.01 | 1.67 ± 1.37^{d} | 7.17 ± 3.06^{d} | 3.00 ± 3.52^{d} |
| | 0.03 | 0.83 ± 1.17^{d} | 5.17 ± 4.45^{d} | 0.17 ± 0.41^{d} |
| Ondansetron | 0.01 | 11.50 ± 5.51 | 7.67 ± 2.25^{d} | 5.83 ± 4.31 |
| | 0.03 | 8.17 ± 2.79 | 14.67 ± 4.68 | 3.50 ± 2.07^{c} |
| | 0.1 | 4.83 ± 2.48^{d} | 12.67 ± 3.45 | 5.33 ± 4.68 |
| | 0.3 | 1.33 ± 0.82^{d} | 5.83 ± 4.45^{d} | 4.67 ± 1.63^{c} |

Oral Antiemetic

| | | | | |
|---|---|---|---|---|
| Vehicle control | 0 | 14.17 ± 3.82 | 14.50 ± 5.54 | 9.50 ± 7.15 |
| Palonosetron | 0.003 | 12.83 ± 6.68 | 5.83 ± 1.47^{d} | 4.50 ± 3.51^{c} |
| | 0.01 | 6.83 ± 4.92^{d} | 6.67 ± 3.83^{d} | 2.83 ± 1.94^{d} |
| | 0.03 | 0.33 ± 0.52^{d} | 3.33 ± 4.55^{d} | 0.33 ± 0.82^{d} |
| | 0.1 | 0.00 ± 0.00^{d} | 1.00 ± 1.67^{d} | 0.00 ± 0.00^{d} |
| Ondansetron | 0.03 | 10.83 ± 2.72 | 9.83 ± 7.52 | 5.67 ± 2.50 |
| | 0.1 | 6.17 ± 5.35^{d} | 10.33 ± 4.84 | 8.50 ± 3.15 |
| | 0.3 | 3.00 ± 3.03^{d} | 4.20 ± 1.92^{d} | 2.83 ± 3.71^{d} |
| | 1 | 0.17 ± 0.41^{d} | 1.50 ± 1.38^{d} | 0.00 ± 0.00^{d} |

^{a}N = Six per treatment group, except in the group receiving 0.3 mg/kg ondansetron po and actinomycin D, in which one dog was dropped due to a dosing error.
^{b}Vehicle control was distilled water in each instance.
^{c}Significantly less than vehicle control, p < 0.05.
^{d}Significantly less than vehicle control, p < 0.01

Under the conditions of these experiments, both palonosetron and ondansetron, whether administered by the intravenous or oral route, reduced the emetic responses to all three antineoplastic agents. The antiemetic effects of both drugs were generally dose-related and palonosetron was consistently at least 10 times more potent than ondansetron.

### Example 5

### Human Trials Assessing the Effectiveness of Single Intravenous Doses of Palonosetron for the Prevention of Highly Emetogenic Chemotherapy-Induced Nausea and Vomiting

A randomized, double-blind, multicenter, dose-ranging Phase II trial was performed to identify the dose response relationship among single I.V. doses of palonosetron. Patients receiving highly emetogenic chemotherapy including cyclophosphamide (>1100 mg/m2) and cisplatin (>70 mg/m2), commonly associated with delayed emesis, were assigned to one of five dose groups of a single I.V. administration of palonosetron. Palonosetron was administered alone (without dexamethasone) as a 30 second intravenous injection, 30 minutes prior to chemotherapy administration. The primary endpoint was 24-hour complete responses (no emesis, no rescue) (CR). Secondary endpoints included complete control (no emesis, no rescue, mild nausea) (CC) and 5-day CR.

161 patients (32 women, 129 men) participated. Key efficacy parameters and results are summarized in Table 6 below. A majority (83.9%) of adverse events were either mild or moderate and not attributed to study medication (86.0%). The most commonly reported adverse events related to study medication include: headache (19.3%); constipation (8.7%), dizziness (2.5%) and abdominal pain (2.5%). No serious drug-related events were reported.

The results demonstrate that in these patients, palonosetron was safe and effective in treating acute emesis and maintained activity through day 5.

TABLE 6
Responders by Dose

| | Palonosetron Dose (mcg/kg) | | | | |
|---|---|---|---|---|---|
| | 0.3-1 | 3 | 10 | 30 | 90 |
| Parameters | (n = 29) | (n = 24) | (n = 25) | (n = 24) | (n = 46) |
| % CR(24 hr) | 24 | 46 | 40 | 50* | 46 |
| % CC(24 hr) | 24 | 39 | 40 | 48 | 46 |
| % CR(Day5) | 17 | 17 | 32 | 33 | 20 |

*Statistically significant differences (p < 0.05) vs. lowest dose group

### Example 6

### Intravenous Formulation

Table 7 below presents a representative formulation of palonosetron formulated for intravenous administration.

TABLE 7

| Ingredient | Mg | |
|---|---|---|
| Palonosetron HCI | 10-100 | mg. |
| Dextrose Monohydrate | q.s. to make | |
| Isotonic | | |
| Citric Acid Monohydrate | 1.05 | mg. |
| Sodium Hydroxide | 0.18 | mg. |
| WFJ | To 1.0 | ml. |

Throughout this application, various publications are referenced. The disclosures of these publications in their entireties are hereby incorporated by reference into this application in order to more fully describe the state of the art to which this invention pertains.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1) A method of treating chemotherapy or radiotherapy-induced acute and delayed emesis comprising administering to an animal a treatment-effective amount of palonosetron.

2) The method of claim 1 wherein about 0.25 mg of palonosetron is administered.

3) The method of claim 1 wherein said palonosetron is administered intravenously.

4) The method of claim 1 wherein said palonosetron is administered intravenously, further comprising admixing said palonosetron with dextrose injection, NaCl, lactated Ringer's solution, or Ringer's solution.

5) The method of claim 1 wherein said palonosetron is administered via Y-site infusion, further comprising admixing said palonosetron with dextrose injection, NaCl, lactated Ringer's solution, or Ringer's solution.

6) The method of claim 1 wherein said palonosetron is administered orally.

7) The method of claim 1 comprising, in any order:
a) administering to the animal from about 3 to about 10 ug/kg of palonosetron; and
b) administering to the animal an emesis inducing amount of a chemotherapeutic agent.

8) The method of claim 7 wherein steps (a) and (b) are performed in sequence.

9) The method of claim 7 wherein step (a) is performed about 30 minutes prior to step (b).

10) The method of claim 1 wherein said palonosetron is administered as an intravenous bolus over a time period of about 10-30 seconds.

11) The method of claim 1 wherein about 0.25 mg of palonosetron is administered in a sterile intravenous solution comprising palonosetron at a concentration of about 0.05 mg/ml.

12) The method of claim 1 wherein about 0.25 mg of palonosetron is administered in a sterile intravenous solution comprising palonosetron at a concentration of about 0.05 mg/ml, wherein said palonosetron comprises an age of from about one month to about two years.

13) The method of claim 1 wherein said palonosetron is administered from a single unit dose ampule comprising about 0.25 mg of palonosetron in a sterile injectable solution.

14) The method of claim 1 wherein said animal is a human.

15) A method of treating chemotherapy or radiotherapy-induced emesis in an animal comprising administering a dose of 0.25 mg of palonosetron to the animal.

16) The method of claim 15 wherein the chemotherapy is defined by successive daily administrations of a chemotherapeutic agent, wherein the method comprises administering 0.25 mg. of palonosetron for two or more successive days.

17) The method of claim 15 wherein the chemotherapy is defined by the administration of a single dose of a chemotherapeutic agent and acute and delayed emesis during a cycle of chemotherapy, wherein the method comprises administering 0.25 mg. of palonosetron for two or more successive days.

18) The method of claim 15 wherein said palonosetron is administered intravenously.

19) The method of claim 15 wherein said palonosetron is administered orally.

20) The method of claim 15 wherein said palonosetron is administered as an intravenous bolus over a time period of about 10-30 seconds.

21) The method of claim 15 wherein said palonosetron is administered in a sterile intravenous solution comprising palonosetron at a concentration of about 0.05 mg/ml.

22) The method of claim 15 wherein said palonosetron is administered in a sterile intravenous solution comprising palonosetron at a concentration of about 0.05 mg/ml, wherein said palonosetron comprises an age of from about one month to about two years.

23) The method of claim 15 wherein said emesis is delayed emesis.

24) The method of claim 15 wherein said palonosetron is administered from a single unit dose ampule comprising about 0.25 mg of palonosetron in a sterile injectable solution.

25) The method of claim 15 wherein said animal is a human.

26) A method of preventing chemotherapy or radiotherapy induced emesis comprising:
a) in a first chemotherapeutic or radiotherapy session:
i) administering to a human a first amount of palonosetron; and
ii) administering to said human an emesis inducing amount of a chemotherapeutic agent or radiotherapy;
b) assessing the effectiveness of the palonosetron; and
c) in a subsequent chemotherapeutic or radiotherapy session, if said human is at least partly non-responsive to said palonosetron in said first chemotherapeutic session, administering to said animal a therapeutically effective amount of a second anti-emetic compound; and
d) wherein the subsequent chemotherapeutic session is performed without an intervening chemotherapeutic session in which a second amount of palonosetron higher than the first amount is administered.

27) A method of preventing chemotherapy or radiotherapy induced emesis comprising, in one chemotherapeutic or radiotherapy session:
a) administering to a human a first dose of palonosetron; and
b) administering to said animal an emesis inducing amount of a chemotherapeutic agent or radiotherapy;
c) assessing the effectiveness of the palonosetron; and
d) if said human is at least partly non-responsive to said first dose of palonosetron, not administering a second dose of palonosetron for at least 24 hours after step (b).
